(19) United States
(12) Patent Application Publication
(10) Pub. No.: US 2007/0207073 A1
(43) Pub. Date: Sep. 6, 2007

(54) Apparatus for supporting and disinfecting a handheld instrument and/or a portion of the user's hand

(76) Inventor: Tod H. Drucker, Maple Glen, PA (US)

Correspondence Address: MCDERMOTT WILL & EMERY LLP, 600 13TH STREET, N.W., WASHINGTON, DC, 20005-3096, US

(21) Appl. No.: 11/582,985

(22) Filed: Oct. 19, 2006

Related U.S. Application Data
(60) Provisional application No. 60/778,693, filed on Mar. 3, 2006.

(51) Int. Cl. A61L 2/00

(52) U.S. Cl. 422/292; 422/300

(57) ABSTRACT

A disinfecting device for a handheld instrument is provided. The disinfecting device includes a receptacle having a cavity for supporting a portion of the handheld instrument, a germicide comprising an amount effective to disinfect the handheld instrument, and a germicidal carrier such that a portion of the handheld instrument contacts the germicide when the portion of the handheld instrument is supported in the receptacle. A disinfecting device for a fingertip is also provided. The disinfecting device includes a germicide effective to disinfect the fingertip, a support base having a cavity within the support base for containing the germicide, and a germicidal carrier for retaining germicide such that at least a portion of the fingertip contacts the germicide upon touching the germicidal carrier.

### RELATED APPLICATION

This application has been assigned application Ser. No. 11/582,985, was filed Oct. 19, 2006, and claims priority to Provisional Patent Application No. 60/831,292, filed on Jul. 16, 2006, and Provisional Application No. 60/778,693, filed on Mar. 3, 2006, which are all incorporated herein by reference.

### FIELD OF THE INVENTION

The present invention relates to a disinfecting device for a handheld instrument and/or a portion of the user's hand. The present invention has particular applicability to a device for disinfecting a writing instrument, keypad, screen, button panel assembly, or restroom door hardware.

### BACKGROUND ART

In recent years, the general population has given closer scrutiny and concern to cross-contamination of public surfaces due to the increased possibility of the transmission of bacteria, viruses and infectious diseases. This is particularly true in public places, where people unknowingly come in direct contact with potentially harmful fluids or solids. Whereas the public is already alerted to the incidence of cross transmission by various media, it is desired to seek further means to reduce the possible consequences of touching exposed public surfaces. Already, public restrooms employ measures to reduce or eliminate touching common surfaces (e.g. infrared faucets sensors and motorized towel dispensers). The concern is growing and other measures are appearing to offset the public concern over similar scenarios (e.g. washing and disinfecting shopping cart handles between uses). Recently, the growing concern over avian flu pandemic will likely increase concern regarding the possibility of accidental transmission of dangerous infectious diseases.

A variety of penholder devices are well known and disclosed in the following publications, e.g., in U.S. Pat. No. 64,610; U.S. Pat. No. 1,463,805; U.S. Pat. No. 1,620,529; U.S. Pat. No. 1,842,677; U.S. Pat. No. 2,150,065; U.S. Pat. No. 3,570,284; U.S. Pat. No. 4,331,721; U.S. Pat. No. 4,678,206; U.S. Pat. No. 4,984,379; U.S. Pat. No. 5,123,548; U.S. Pat. No. 5,839,712; U.S. Pat. No. 6,167,596; and U.S. Pat. No. 6,773,188.

Furthermore, a variety of chemical compositions for sterilizing, disinfecting, pathogenic inactivation, etc., are well known and described in the following publications, e.g., in US 2005/0239671; US 2004/0209790; US 2004/0033916; U.S. Pat. No. 6,927,237; U.S. Pat. No. 6,797,681, U.S. Pat. No. 6,762,161; U.S. Pat. No. 6,489,276; U.S. Pat. No. 6,387,856; U.S. Pat. No. 6,043,207; U.S. Pat. No. 5,810,944; and U.S. Pat. No. 5,663,132.

Moreover, a variety of sterilization devices are disclosed for medical and dental instruments in the following publications, e.g., in U.S. Pat. No. 4,308,229; U.S. Pat. No. 5,480,302; U.S. Pat. No. 5,407,354; U.S. Pat. No. 5,447,684; and U.S. Pat. No. 6,224,826.

At present, writing instruments remain handheld devices and subsequently may be contaminated by the operator. In most retail locations and professional offices pens are inevitably, or by design, shared instruments. Further, most electronic credit card processing readers employ an electronic pen-like instrument to record the purchaser's signature as required. The electronic devices are shared among users and are necessary to record the signature and finalize the financial transaction and do not allow the user to substitute a personal writing instrument.

Commercial transactions routinely require the use of keypads and/or touch screens. Elevators routinely employ a panel of buttons for floor selection. Similarly, most restrooms utilize a pull handle or push plate to exit the restroom. These devices and fixtures are shared by multiple users and may become readily contaminated.

### SUMMARY OF THE INVENTION

The disinfecting device of the present invention addresses these problems with conventional handheld instruments by serving to disinfect the handheld instrument between uses, including the finger(s) or hand before or after use of a keypad and/or touch screen device, and/or button panel assembly, and/or restroom fixture, and can be a cost effective and disposable or refillable device. Currently, mechanical, touch screen and biometric devices require that a sequence of users physically contact the same, potentially contaminated, interface. An advantage of the inventive disinfecting device is that it increases user confidence that they do not risk contamination by making purchases at any site utilizing the disinfecting device of the invention, nor any office utilizing the disinfecting device of the invention. An additional advantage of the present invention is that the inventive device is applicable to minimizing cross-contamination of other shared fixtures or surfaces.

According to the present invention, the foregoing and other advantages are achieved in part by an apparatus for supporting and disinfecting a handheld instrument, comprising (a) a receptacle, having a cavity, for supporting a handheld instrument; (b) a germicide; and (c) a germicide carrier, for carrying the germicide, disposed within the cavity such that a portion of the handheld instrument contacts the germicide carrier and the germicide when the handheld instrument is supported by the receptacle.

Another aspect of the present invention is an apparatus comprising a base having a cavity and an opening at a surface of the base; a germicide disposed in the cavity; and a germicide delivery device to deliver the germicide to the opening for contacting a portion of a hand and for disinfecting the hand portion with the germicide.

Additional advantages of the present invention will become readily apparent to those skilled in this art from the following detailed description, wherein only exemplary embodiments of the present invention are shown and described, simply by way of illustration of the best mode contemplated for carrying out the present invention. As will be realized, the present invention is capable of other and different embodiments, and its several details are capable of modifications in various obvious respects, all without departing from the invention. Accordingly, the drawings and description are to be regarded as illustrative in nature, and not as restrictive.

### BRIEF DESCRIPTION OF THE DRAWINGS

Reference is made to the attached drawings, wherein elements having the same reference numeral designations represent like elements throughout, and wherein:

FIG. 1 is a perspective view of an apparatus for disinfecting a portion of a hand according to an embodiment of the present invention.

FIG. 2a is a cross sectional perspective view of the apparatus of FIG. 1.

FIG. 2b is a cross sectional perspective view of an apparatus according to another embodiment of the present invention.

FIG. 3 is a cross sectional view of an apparatus according to another embodiment of the present invention.

FIG. 4 is an external view of an embodiment of a holder for the apparatus of FIGS. 1-3.

FIG. 5 is an external view of the holder (depicted in FIG. 4) in situ with the apparatus of FIG. 1.

FIG. 6a is a perspective view of a stylus device and base according to the present invention.

FIG. 6b is another perspective view of the stylus device and base of FIG. 6a.

FIG. 7 is a front view of the stylus device of FIG. 6a.

FIGS. 8a-8c are cross sectional views of the stylus device of FIG. 6a.

FIG. 9 is a perspective view of another embodiment of the stylus device according to the present invention.

FIG. 10 is a front view of the stylus device of FIG. 9.

FIG. 11a is a cross sectional view of a stylus device according to an embodiment of the present invention.

FIG. 11b is a cross sectional view of a stylus device according to an embodiment of the present invention.

FIG. 12a is a perspective view of a stylus device, base, and refill container according to the present invention, in situ.

FIG. 12b is a cross sectional perspective view of a stylus device, base, and refill container according to the present invention, in situ.

FIG. 12c is a cross sectional view of a stylus device, base, and refill container according to the present invention, in situ.

FIG. 13 is a cross sectional view of a stylus device, base, and refill container according to the present invention, in situ.

### DETAILED DESCRIPTION OF THE INVENTION

The description that follows is presented to enable one skilled in the art to make and use the present invention, and is provided in the context of a particular application and its requirements. Various modifications to the disclosed embodiments will be apparent to those skilled in the art, and the general principals discussed below may be applied to other embodiments and applications without departing from the scope and spirit of the invention. Therefore, the invention is not intended to be limited to the embodiments disclosed, but the invention is to be given the largest possible scope which is consistent with the principals and features described herein.

It will be understood that in the event parts of different embodiments have similar functions or uses, they may have been given similar or identical reference numerals and descriptions. It will be understood that such duplication of reference numerals is intended solely for efficiency and ease of understanding the present invention, and are not to be construed as limiting in any way, or as implying that the various embodiments themselves are identical.

One embodiment of the disinfecting device of the present invention comprises: (i) a receptacle having a cavity within the receptacle for supporting the handheld instrument, (ii) a germicide comprising an amount effective to disinfect the handheld instrument, and (iii) a germicidal carrier such that a portion of the handheld instrument contacts the germicide when the portion of the handheld instrument is supported in the receptacle, and (iv) an area containing germicide such that an extremity of the user contacts the germicide when the device is in use. The receptacle is either fixably attached to a base or it contains a means for removably attaching the receptacle to a base or stand, e.g. by Velcro®. In this embodiment the base is fixed to a horizontal surface.

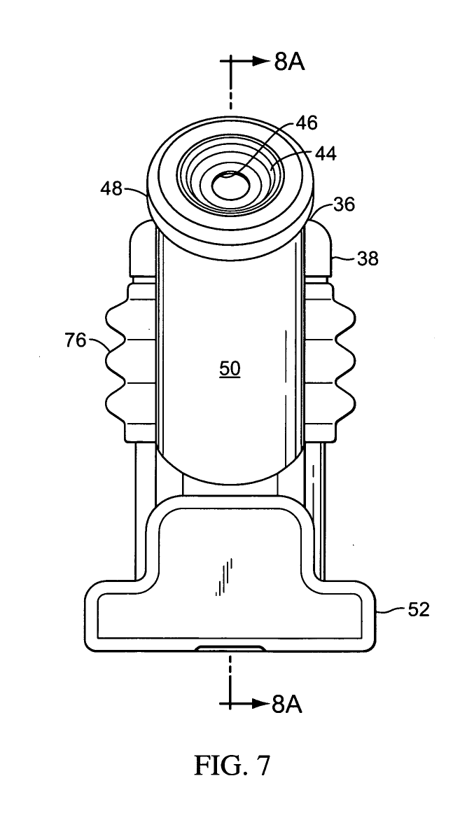

In one embodiment, as shown in FIGS. 6a, 6b, 7, and 8a, the outer stylus tube 50 is cylindrical and is fixably attached to an interlocking detachable base 52, the device is further secured to the base via a base retention clip 54. The longitudinal channel for stylus entry and exit 56 for the handheld instrument is contained within the outer stylus tube 50.

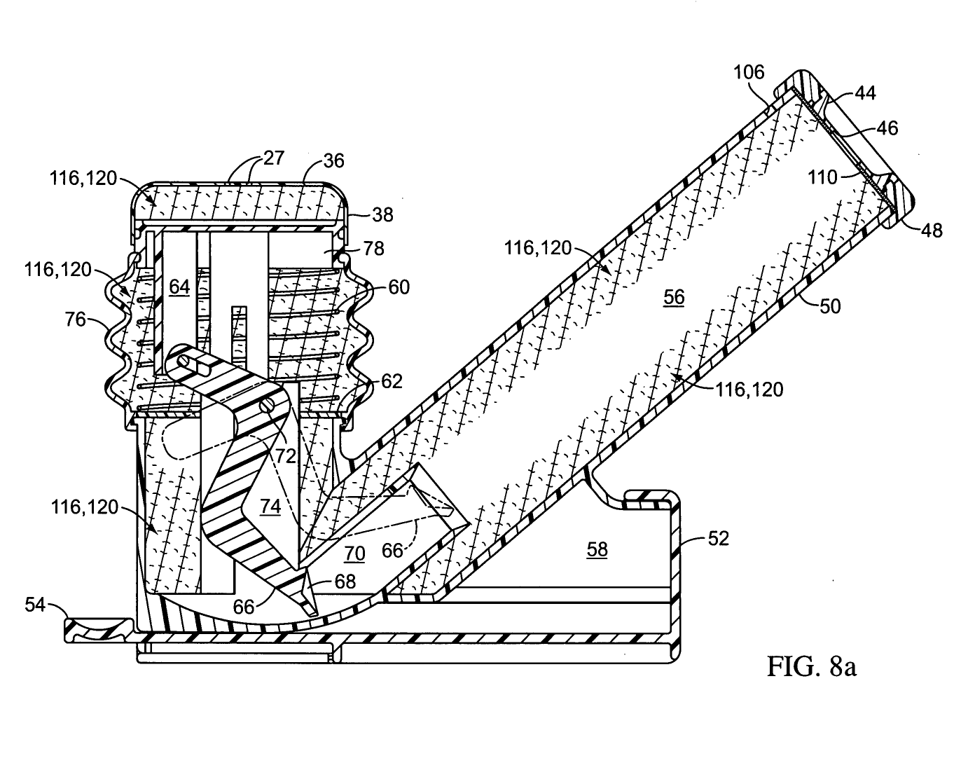

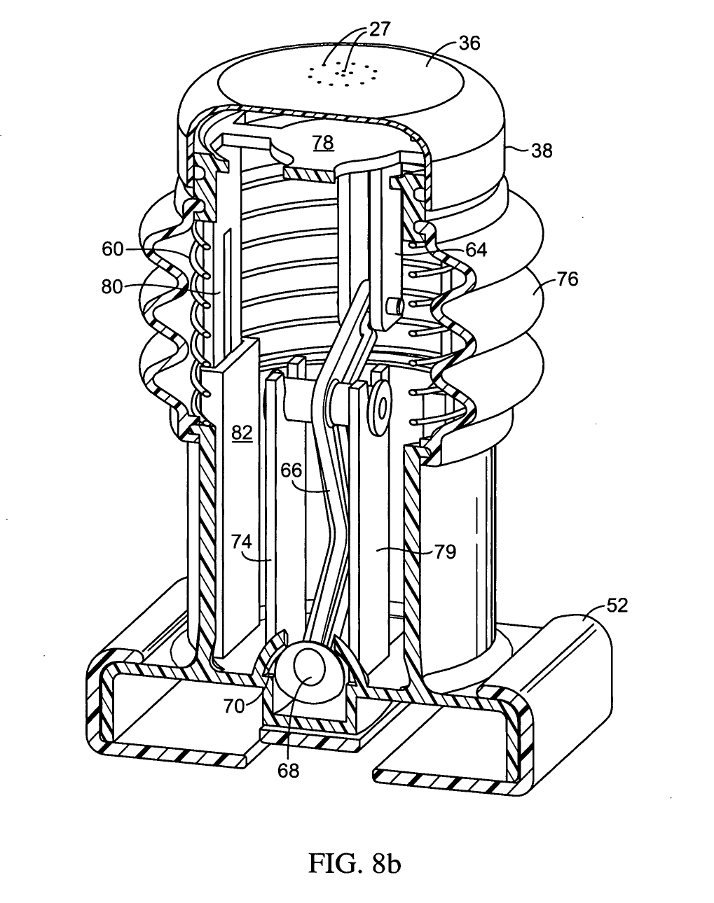

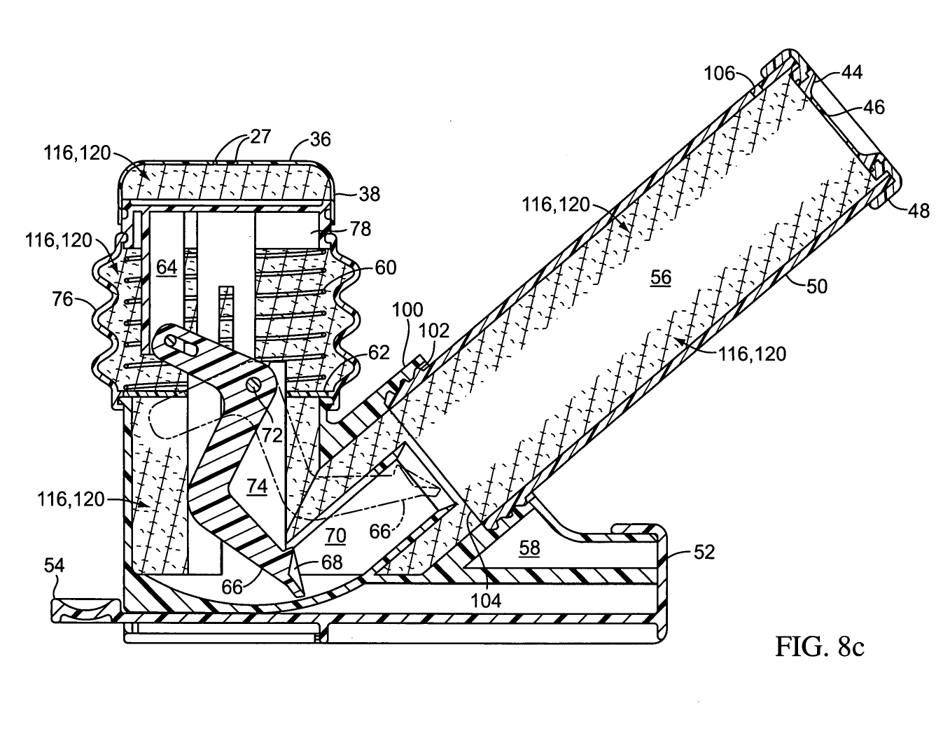

A flexible elastomeric or polymeric material diaphragm 44, having a central opening 46, is secured to the outer stylus tube 50 by an outer retention cap 48. FIG. 8a illustrates a modification to this portion of the device, as represented by 110. This component 110, located beneath the elastomeric diaphragm 44, and further held in place by the retention cap 48, may include, but not be limited to, the addition one or more of: (a) a ring or disk of abrasive material, e.g. Scotchbrite®; (b) a ring or disk of scent producing material; (c) a ring or disk of color producing material; or (d) a ring or disk of other desirable properties,

As represented in FIGS. 8a and 8b, an external membrane 36 is secured to the absorbent material platform 78 by an outer retention ring 38. The external membrane 36 is the area at which the device is operated by downward pressure, thus compressing the bellows 76 and spring 60. The external membrane 36 is preferentially perforated 27 in its central portion to permit the flow of germicide 116 through the membrane. In preferred application the membrane would be a nonabsorbent, non-reactive material and feel slippery or slick to the user. PolyTetraFluoroEthylene (PTFE) based materials e.g. Teflon®, Gore-Tex®, or Teflon/silicone materials, e.g. Silon® are envisioned as likely membrane materials but are selectively permeable materials and would be additionally perforated to permit adequate flow of germicide 116 past the membrane interface.

As further represented in FIG. 8a and FIG. 8b, the device is essentially filled with absorbent material 120, excepting a tubular, longitudinal channel for stylus entry and exit 56. The device is further filled with absorbent material 120 in all areas beneath the external membrane 36 excepting the interlocking detachable base 52, and also excepting those specific areas which would directly interfere with the mechanical action of the internal parts described herein.

The absorbent material 120 is placed so as to contact, permit, and facilitate wicking and absorption of any germicide 116 within the device. The absorbing material comprises a prefabricated, shaped block(s) of material, or individual strips, or segments. To facilitate construction and manufacture, the absorbent material 120 is inserted, for example, as a shaped composite of shredded, granulated, laminated, layered, and/or pleated material and can include any additional barriers or laminations deemed advantageous to the performance of the device. Examples of absorbing materials that are used in the present invention include one or more compressed layers of cellulosic fluff pulp that may be in combination with superabsorbents (polymers having the capacity to absorb water or body liquids many times their own weight). Other types of absorbent structures are airlaid cellulosic fibrous webs which have been bound with a bonding agent, e.g. latex, heat meltable bonding fibers or the like, dry formed reel pulp, absorbent foam materials, etc. thereby causing evaporation of the germicide 116 from the wick or absorbent material 120.

It is desired that the absorbing material 120 contact the germicide 116 so as to provide constant absorption of the germicide 116 by the absorbing material 120 and that the absorbing material 120 be capable of continuous absorption of the germicide 116 through evaporation of the germicide 116 as a result of the evaporation holes 106.

The absorbing material is shaped such that it is in intimate contact with those desired portions of the handheld instrument, e.g., where the barrel of the handheld instrument has a circular width, the absorbing material would encircle the desired portions of the barrel. It is desirable for the absorbing material to have a desired degree of compression and resiliency to provide the necessary opening to accommodate the diameter of the handheld instrument when inserted. The absorbing material extends inside of the upper portion of the outer stylus tube 50 along a given length and terminates in a reservoir of germicide 116 agent. The absorbent material 120 immediately lining the central stylus channel 56 may employ polyethylene terephthalate (PET) fibers or other hydrophilic fibers useful for scrubbing.

In practical use, at rest, the body of a writing instrument or stylus (not shown) would be situated within the longitudinal channel 56, and the tip of the writing instrument or stylus would reside within the stylus receptacle chamber 70. In this position the writing instrument or stylus is in general, intimate contact with the absorbent material 120 and the germicide 116. Any remaining portion of the stylus not, perhaps, in intimate contact with the absorbent material 120 and germicide 116 would, nevertheless, be exposed to the vapor pressure of the surrounding germicide 116.

The external membrane 36 contains beneath it an absorbing material such that the finger(s) or hand contacts the germicide 116 expressed through the perforations 27 upon pressing external membrane 36.

As the external membrane 36 is pressed, it compresses germicide 116 laden absorbent material 120 between the external membrane 36 and the absorbent material platform 78. FIG. 8b best shows that the absorbent material platform 78 is perforated with a series of openings in which absorbent material 120 is able to wick germicide 116 from within the body of the device to beneath the external membrane 36.

The downward force is further transmitted upon the flexible bellows 76, the spring 60 (supported by the spring support platform 62), lever actuator struts 64 and the lateral support struts 80. The lateral support struts 80 descend into the lateral support strut housings 82. The lever actuator strut 64 engages the lever 66. The lever 66 is positioned atop and between a pair of lever pin support struts 74 and further supported via an integrated lever pivot pin 72. The downward force drives down the rear arm of the lever 66 while simultaneously raising the front arm of the lever 66 and specifically driving the stylus tip receptacle 68 through the stylus receptacle chamber 70. The upper aspect of the stylus receptacle chamber 70 is slotted to permit free movement along this specific path. Concurrent with the described movement of the stylus tip receptacle 68 through the stylus receptacle chamber 70, the writing instrument or stylus would be partially ejected from the device. Further, as the user's finger(s) or hand exerts downward force it compresses the absorbent material 120 within the flexible bellows 76. Additional germicide 116 would be released and exude though the perforations 27 in the external membrane 36—effectively a “pumping” action—and further wet the finger(s) and/or any other portion of a user'hand with germicide 116. Additional wetting of the external membrane 36, if desired, could be accomplished by a) adding additional perforations to the external membrane 36, and/or b) adding a common mechanical foam dispenser within the bellows 76 and beneath the external membrane 36. Whereby many transaction situations require the use of keypads and/or touch screen interaction in addition to using stylus devices, the user's wetted finger(s) or hand portion is thus adequately prepared to touch these surfaces, i.e. a keypad or touch screen, without contaminating said surface, nor becoming contaminated itself.

Once the writing instrument or stylus is sufficiently ejected from the device, the user would release the downward force upon the external membrane 36, and the flexible bellows 76 and spring 60 (seated upon the spring support platform 62) would resume their prior shape and return the depressible portion of the device to its prior position.

As represented in FIGS. 6a, 6b, 7, 8a, 8b, 8c, 9, 10, 11a, and 11b the device in these embodiments is disposable as a whole, excepting the interlocking base 52. The device is restrained into the base 52 by the base retention clip 54.

Most germicides are volatile, and a series of evaporation holes 106 surrounding the perimeter of the outer retention cap 48 enhance the wicking and evaporation actions. Packaging of the device could be designed such that these evaporation holes 106 are necessarily exposed when the device is removed from its external packaging. Additionally, the precise design of this series of evaporation holes 106 could be modified and configured as desired or needed to require replacement of the device at a predetermined interval.

FIG. 8c represents an additional embodiment utilizing the flexible bellows 76 in which the outer stylus tube 50, the elastomeric diaphragm 44, and the outer retention cap 48 constitute a threaded “cartridge” assembly prefilled with germicide 116. Said “cartridge” assembly is joined to the remainder of the device via the externally threaded cartridge coupling 102 and the internally threaded base coupling 100. A removable sealing film 104 prevents loss of germicide 116 until the “cartridge” assembly is installed and threaded via the coupling arrangement 100, 102. In use, additional germicide 116 could optionally be added within the remaining bellows portion of the device prior to installing the “cartridge” assembly.

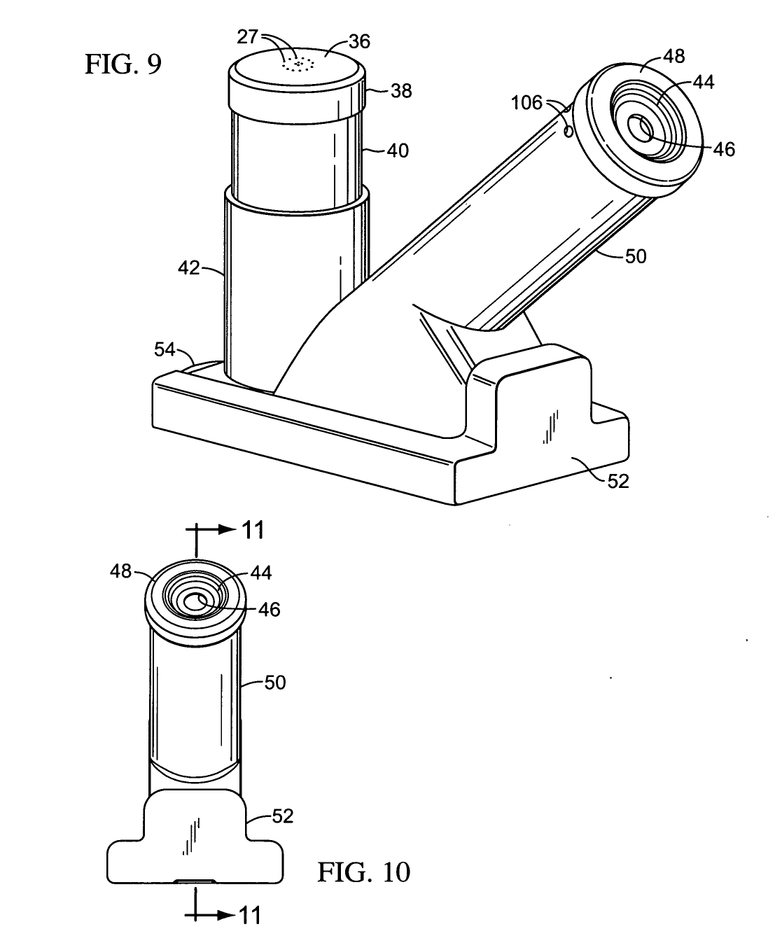

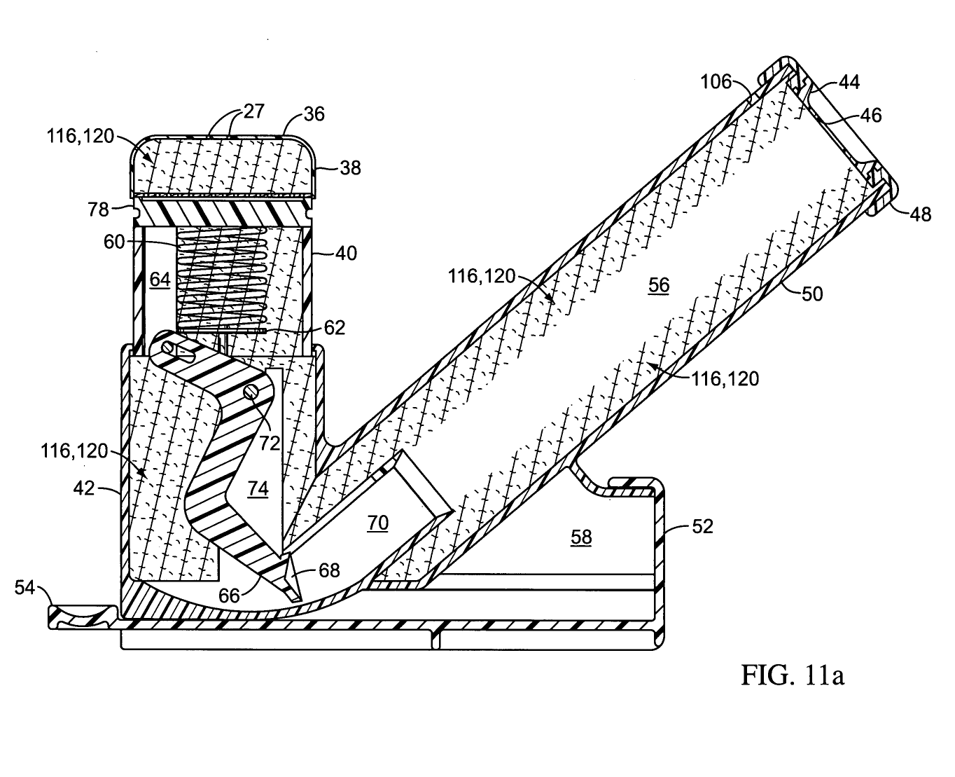

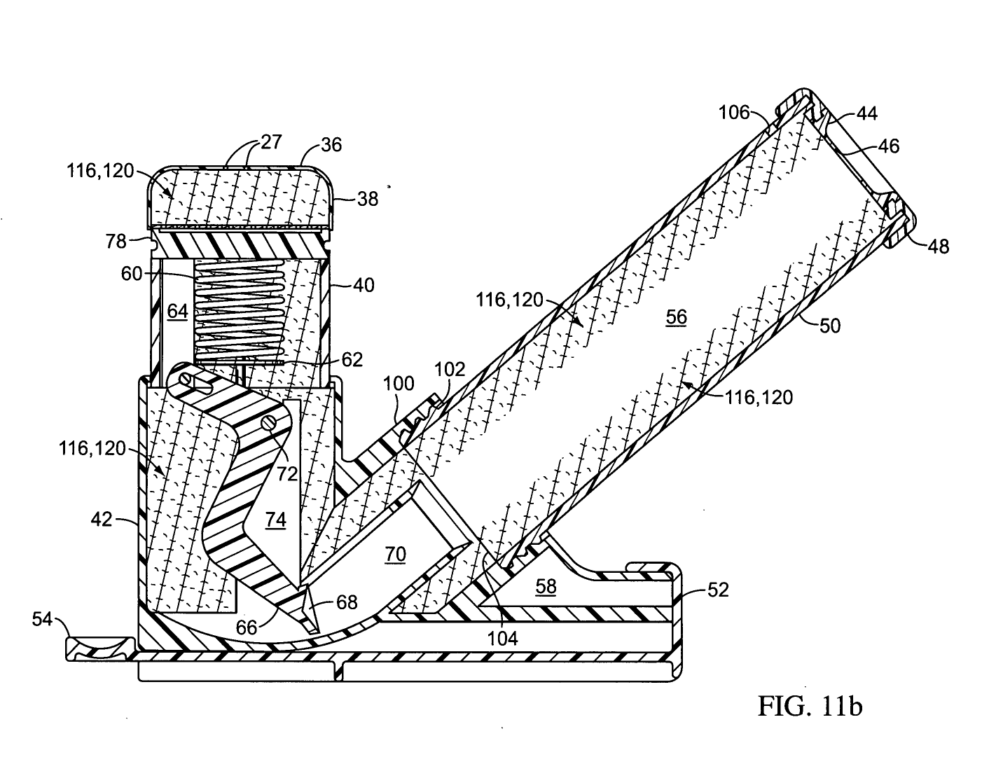

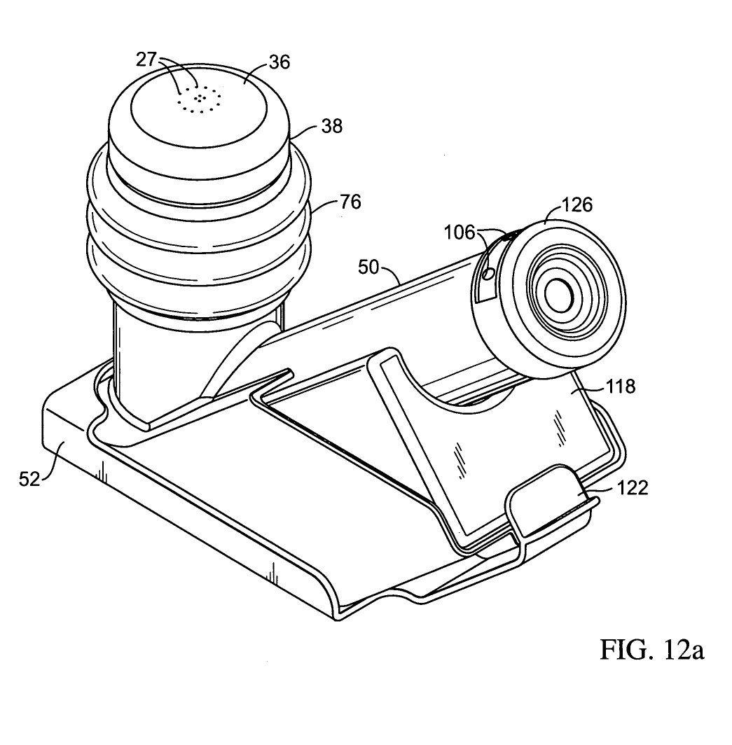

FIG. 9, FIG. 10 and FIG. 11 a show another embodiment in which the resilient flexible bellows 76 is replaced by a piston mechanism employing an inner 40 and outer 42 wall of the piston cylinder.

FIG. 11b represents another embodiment utilizing a piston mechanism (employing an inner 40 and outer 42 wall of the piston cylinder) in which the outer stylus tube 50, the elastomeric diaphragm 44, and the outer retention cap 48 constitute a threaded prefilled “cartridge” assembly with germicide 116. Said “cartridge” assembly is joined to the remainder of the device via the externally threaded cartridge coupling 102 and the internally threaded base coupling 100. A removable sealing film 104 prevents loss of germicide 116 until the “cartridge” assembly is installed and threaded via the coupling arrangement 100, 102. In use, additional germicide 116 could optionally be added to the remaining piston assembly of the device prior to installing the “cartridge” assembly.

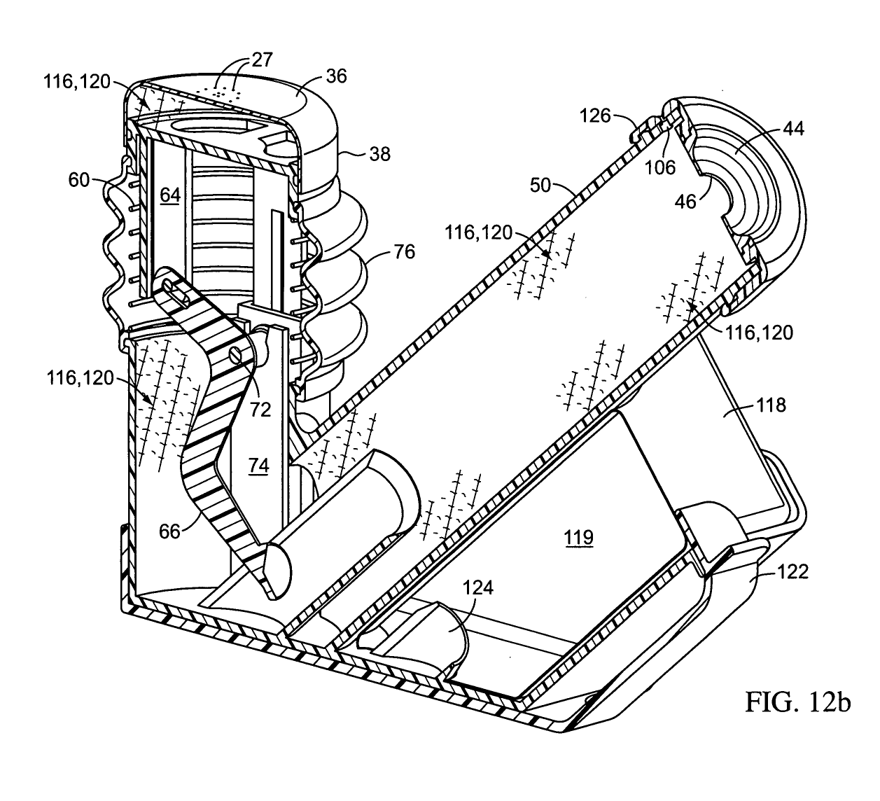

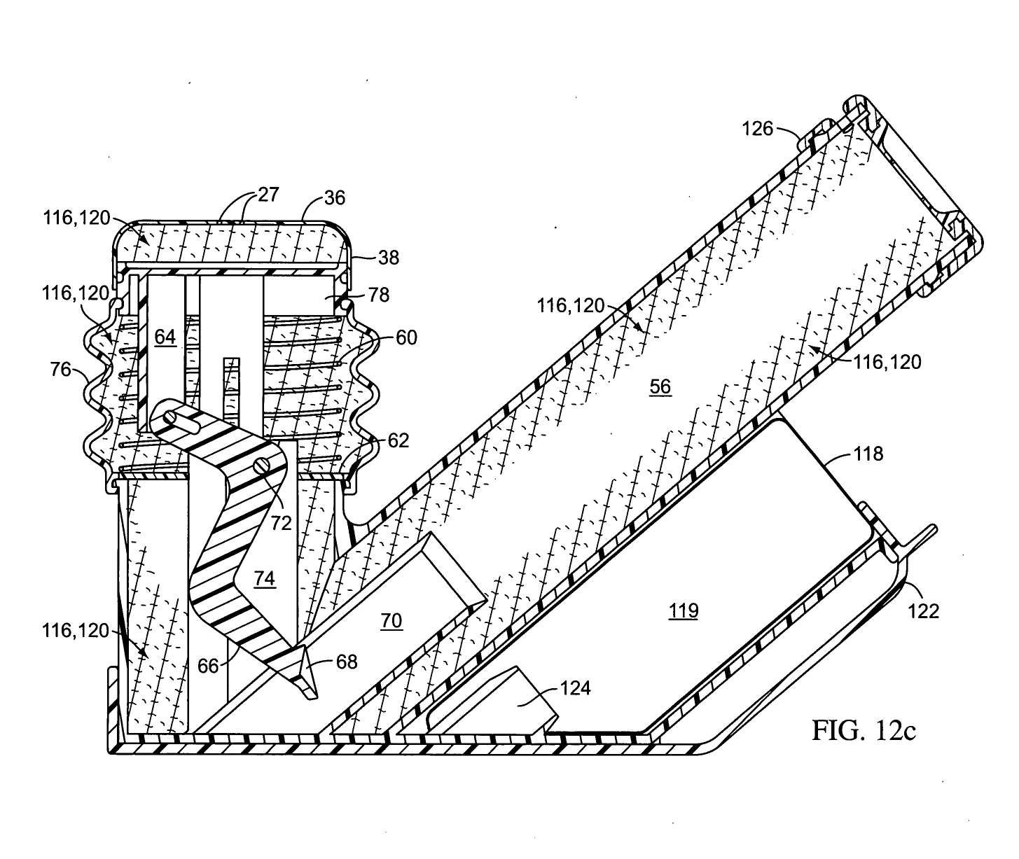

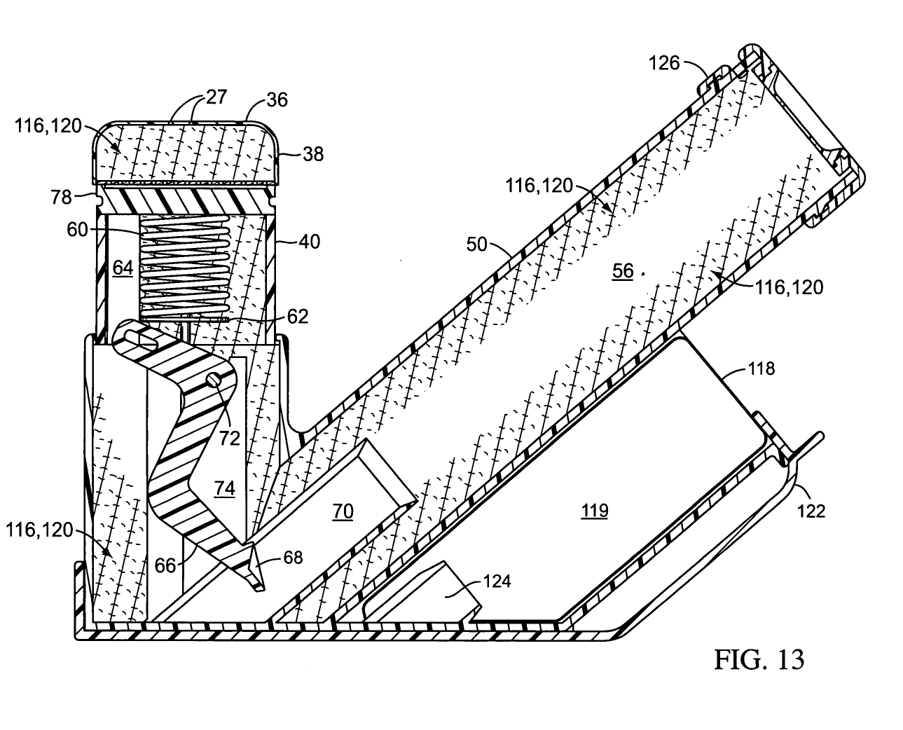

FIGS. 12a, 12b, 12c, and 13 depict embodiments which include the ability to refill the device. These embodiments have the feature that the germicide is delivered in a renewable “box” or “pouch” to the device. Further, they may contain a means to visually indicate whether adequate levels of germicide are present.

When a removable external container 118 containing the additional germicide 119 is inserted into the device, a punch 124 contacts and perforates a “designed to fail” puncture spot on the removable external container 124 which releases the additional germicide 119. There is sufficient communication for the newly released additional germicide 119 to contact the absorbent material 120. The removable external container 118 and additional germicide 119 together constitute a refill. The refill is held in place by a refill retention clip 122.

These embodiments further illustrate a circumferential retention cap and ring 126 overlying the evaporation holes 106. This retention ring and cap 126 may rotate around the long central axis of the outer stylus tube 50. A portion of the outer edge of the retention cap and ring 126 may have openings or slots which, via rotation, could selectively expose or variably occlude the underlying evaporation holes 126, and thereby further regulate the subsequent evaporation of the germicide 116 within the device. A removable cap or covering could be applied over one or more of: (i) the central opening 46, (ii) evaporation holes 106, and/or (iii) retention cap and ring 126 during periods of inactivity to further reduce evaporative loss.

It is desired that sufficient germicide 116 be available within the device to appropriately and sufficiently provide the desired level of disinfection. A chamber for electronics and sensors 58 is included to house materials, which optionally, can indicate the current status of the internal germicide 116 and/or the need to replace the device.

In most embodiments, the device is essentially filled with germicide 116, and thus wetting and/or saturating the absorbent material 120. Prior to use of the device, the absorbent material 120 is either dry, wetted with germicide 116, presaturated with germicide 116 or otherwise coated with germicide 116.

The germicide 116 of the present invention has at least one of the following features: (i) a single non-mixed, non-activated liquid; (ii) a activated multi-component mix of separated powder/liquid, or liquid/liquid; (iii) a dry powdered or granulated germicidal agent. The term “germicide” and “germicidal” is understood to be interchangeable with the terms “antimicrobial” and “antibacterial” and other such like terms as would be familiar to one of ordinary skill. The term “germicidal effectiveness” is intended to mean that, given a sufficient amount of germicide, the germs, microbes, bacteria, virus, etc concentration of a sample is decreased by at least about 30% over a desired period of time.

Any germicide capable of providing the desired level of germicidal activity or germicidal effectiveness may be used in the present invention. Examples include antimicrobial agents include ethyl alcohol, carboxylic acids, diacids, or triacids (e.g., formic acid, acetic acid, butyric acid, heptanoic acid, octanoic acid, nonanoic acid, decanoic acid, salicylic acid, mandelic acid, succinic acid, adipic acid, glutaric acid, EDTA and citric acid), carboxylic esters (e.g., p-hydroxy alkyl benzoates and alkyl cinnamates), sulfonic acids (e.g., dodecylbenzene sulfonic acid), iodo-compounds or active halogen compounds (e.g., elemental halogens, halogen oxides, iodine, interhalides, polyhalides, hypochlorite salts, hypochlorous acid, hypobromite salts, hypobromous acid, chloro- and bromo-hydantoins, chlorine dioxide, and sodium chlorite), active oxygen compounds including hydrogen peroxide, a percarbonate, a perborate, a persulfate, or isolated or equilibrium derived or isolated peroxycarboxylic acids such as chloroperbenzoic acids, peroxyacetic acid, peroxyheptanoic acid, peroxyoctanoic acid, peroxydecanoic acid, peroxyformic acid, peroxycitric acid, peroxyglycolic acid, peroxylactic acid, peroxybenzoic acid, and monoester peracids derived from diacids or diesters (e.g., such as adipic, succinic, glutaric, or malonic acid and mixtures thereof), organic peroxides including benzoyl peroxide, alkyl benzoyl peroxides, ozone, singlet oxygen generators, and mixtures thereof, phenolic derivatives (e.g., o-phenyl phenol, o-benzyl-p-chlorophenol, tert-amyl phenol and C₁-C₆ alkyl hydroxy benzoates), quaternary ammonium compounds (e.g., alkyldimethylbenzyl ammonium chloride, dialkyldimethyl ammonium chloride and mixtures thereof), and mixtures of such antimicrobial agents, in an amount sufficient to provide the desired degree of microbial protection. Many antimicrobial agents (e.g., iodophors, peracids, hypochlorites, chlorine dioxide, ozone, etc.) have a broad spectrum of antimicrobial properties. Chemical compositions, for example, described in the following publications are used in the present invention: US 2005/0239671; 2004/0209790; US 2004/0033916; U.S. Pat. No. 6,927,237; U.S. Pat. No. 6,797,681, U.S. Pat. No. 6,762,161; U.S. Pat. No. 6,489,276; U.S. Pat. No. 6,387,856; U.S. Pat. No. 6,043,207; U.S. Pat. No. 5,810,944; and U.S. Pat. No. 5,663,132. Many antimicrobial agents (e.g., iodophors, peracids, hypochlorites, chlorine dioxide, ozone, etc.) have a broad spectrum of antimicrobial properties and can beneficially be used. Hospital grade disinfectants are used, e.g., Hospital Grade A disinfectants. One test for determining degree of disinfection is “TGA Disinfectant Test” Kelsey and Maurer. This test is used to assist in choosing appropriate germicides for use in the invention. It is well within the skill of the art to determine and select the particular germicide that targets time of disinfection and considers effect of materials used in the device.

The germicide 116 usable in the invention varies according to the intended application (e.g., sanitizer, disinfectant, sterilant, aseptic packaging treatment, etc.) and the applicable public health requirements. For example, in one embodiment, germicides that are used in the present invention include those meeting the guidelines as set out in Germicidal and Detergent Sanitizing Action of Disinfectants, Official Methods of Analysis of the Association of Official Analytical Chemists, paragraph 960.09 and applicable sections, 15th Edition, 1990 (EPA Guideline 91-2) (a sanitizer should provide a 99.999% reduction (5-log order reduction) within 30 seconds at room temperature, 25±2° C., against several test organisms). As used herein, germicides include any “disinfectant” referring to an agent that kills all vegetative cells including most recognized pathogenic microorganisms, using the procedure described in A.O.A.C. Use Dilution Methods, Official Methods of Analysis of the Association of Official Analytical Chemists, paragraph 955.14 and applicable sections, 15th Edition, 1990 (EPA Guideline 91-2). Germicide 116 can also include other components, such as emollients or skin softeners. The germicidal agents discussed herein can be used in combination with any dermatologic or cosmetic agents or components deemed beneficial or advantageous.

Antimicrobial and germicidal solutions are either a single component (e.g. Lysol®), an activation required two component powder/liquid (e.g. Maxcide®), or a two component liquid/liquid. Typically, the activation-required mixtures have a designated useful life on the order of 28-30 days once activated. If these types of agents are used with in the invention described, a monitoring capability of the usefulness of the agents is a desired adjunct to the reliability and credibility of the device. Thereby, to ensure effectiveness, a dispose by date label or punch block, e.g., what is typically used on auto batteries, is inscribed by the user upon activation. Alternately, a chemical sensing and color changing monitor strip is included with the device. Alternately, a sensor is located in the device and indicates the need to replace the device using a visual and/or auditory indication or warning. The two solvent compositions typically contain a second solvent that is not or is only sparingly soluble in a diluting solvent. Alternatively, the two solvent compositions form a clear single-phase solution. The two solvent antimicrobial compositions reduce the population of microbes on various surfaces such as facilities, containers, or equipment found in food, beverage, or pharmaceutical industries at temperatures between about −70° C. to about 100° C.

It is desired that the compositions chosen do not corrode or prevent the germicidal device from performing its intended function in regard to the handheld instrument. Compositions non-caustic to plastics, metal, etc. are therefore generally preferred.

In one embodiment, the germicide 116 is also comprised of non-chemical agents such as ultraviolet radiation that are administered by ultraviolet bulbs contained within the receptacle. Alternately, dry germicide(s) 116 is impregnated in the material surrounding the handheld instrument.

The absorbent material 28, 120 comprises a wicking material and it is understood that this absorbent material 28,120 may be any known material which has the ability to absorb fluid, including super absorbent materials. As represented in FIGS. 6 through 11c, the absorbent material 120 is shaped to form or provide a longitudinal channel for stylus entry and exit 56 into which the handheld instrument is placed and contacts the absorbent material 120 and the germicide 116 at all desired areas. The channel for stylus entry and exit 56, within the outer stylus tube 50, can have an arbitrary shape, e.g., square, circular, or triangular. It is generally configured to match a width of the barrel of a handheld instrument to be inserted therein and is generally shaped for contacting the handheld instrument with the germicide.

Certain embodiments of the present invention include combinations of the angled outer stylus tube 50, interlocking detachable base 52, flexible bellows assembly (36,38,60,64,66,68,70,72,74,76,78,80,82), piston assembly (36,38,40,42,60,62,64,66,68,70,72,74,78) and have the following features: (i) self-mounting to a horizontal surface with self-adhesive material (e.g. foam adhesive strips, Velcro® material); (ii) mounting within a durable mounting assembly fixed to a horizontal or vertical surface having the capacity to swivel and mount in proximity to any convenient surface; (iii) a manually operated “ejector” to fully submerge the writing instrument within the outer stylus tube 50; (iv) a motor & IR sensor assembly to automatically retract and eject the writing instrument; (v) either passive evaporation of liquid or active (e.g. fan forced, or heated) evaporation of liquid; (vi) ejection of the writing instrument in a direct straight-line fashion, or (vii) rotation of the writing instrument within the germicide 116 and outer stylus tube 50 to effect greater cleansing and decontamination.

Another embodiment of the invention provides for contacting the germicide with the handheld instrument with an electromechanical sensor and motor, which has a series of germicide sprayers in lieu of absorbing material.

In one embodiment, the outer stylus tube 50 is cylindrical or ovoid in cross section. The device may be extruded, molded or otherwise manufactured to preclude any seam susceptible to leakage of contents. In another embodiment, the device is manufactured from mating portions with seam delineations reliably sealed along their length to ensure security of the germicide 116.

For certain embodiments, the mechanism for advancing and retracting comprises or utilizes a lever, spring, plunger, bellows, piston, mechanical or electromechanical device, hydraulics, suction, etc. In one embodiment, a manual ejector for ejecting, releasing or advancing the handheld instrument is provided. Another embodiment, provides for automatic timed release of the handheld instrument where desired. The ejector is activated by the hand or foot of the operator or other individual. In one embodiment of the present invention, the germicidal carrier has a shape such that germicide 116 contacts virtually all of the outer periphery of the handheld instrument such that the handheld instrument is fully exposed to the germicide 116 via the absorbent material 120 within the outer stylus tube 50 and at least a portion of the handheld instrument then being retracted out of the central opening in diaphragm 46 of the device such that the user of the handheld instrument may withdraw the handheld instrument with minimal and, in some cases, no exposure to any portion of the device except the handheld instrument. This configuration desirably enables each user to touch only a handheld instrument that has been fully exposed to the germicide 116.

The material or materials that are used for the device include any one of or combination of plastic, rubber, hardened polymeric material, etc. Any material commonly used in the industry to make molded materials may be used.

As shown in FIGS. 6, 7, 8a, 8d, 10, 11a, 11c, an outer retention cap 48 includes an elastomeric diaphragm 44 with central opening 46 sized to accommodate the writing instrument. The central opening 46 is circular, square or shaped to accommodate a variety of handheld instrument configurations. In another embodiment, the opening 46 has a series of small radial cuts along the perimeter to provide a comfortable and desirable “feel” when inserting and removing the handheld instrument. One or more additional vent holes 106 are employed within any or all of the (i) cap 48, (ii) the elastomeric diaphragm 44, (iii) the outer stylus tube 50 to allow or promote evaporation of the absorbed germicide 116 and better absorption of the germicide 116 within the absorbent material 120. A removable cover can be fitted over the outer retention cap 48 cap to minimize evaporation during periods of non-use.

As shown in FIG. 6, the device is either fixably or removably attached to a base 52 Alternatively, the base 52 forms a part of the device. The base 52 generally holds the outer stylus tube 50 substantially upright at a variety of angles, particularly, 25 to 90 degrees, more particularly, 30 to 70 degrees, more particularly, 35 to 60 degrees, more particularly, 35 to 50 degrees, to horizontal. The device is configured to hold a portion of germicidal agent 116 or it is hollow or solid and does not contain a reservoir for the germicide 116. When hollow, the outer stylus tube 50 has a longitudinal channel 56 to allow flow of the germicide 116 to the absorbent material 120.

In one embodiment, the device (without any base) self-mounts to a horizontal surface with self-adhesive material, preferably Velcro®-type, to hold the germicidal device of the invention in place. In another embodiment, a separate base 52, properly sized to attach to and/or seize the device, is mounted to a horizontal, vertical, or angled surface to hold the germicidal device in place. In further embodiments, the device is “portable”; i.e., not mounted in place. For example, an easily movable version of the device could be clipped to a shirt pocket, or used at a restaurant table to provide a clean pen to a patron when signing a bill.

The handheld instrument is any handheld instrument such as a writing device, signature device, and indicating device. Examples include pens, markers, and attached electronic wands, e.g. to a credit card signature station, electronic pointing devices, etc. The handheld instrument is contained within the germicidal device of the invention or it is not contained within the germicidal device. Further, it is possible, for example, to use handheld instruments made out of antimicrobials or antimicrobial coatings on the exterior thereof.

The absorbent germicidal carrier material used for contacting the germicide to at least a portion of the handheld instrument is by any means known in the art. For example, one germicidal carrier comprises an absorbing material such as a wicking material. The absorbent material for retaining the germicide could be supported and pre-positioned within a cage assembly, which is inserted within the outer stylus tube 50.

It is desired to use a wicking element to promote a wicking action that absorbs the germicide throughout the portion of the stylus tube 50 that is to be in contact with the handheld instrument. In one embodiment, the absorbing material is an evaporative element. The evaporative element is made of materials and with a configuration sufficient to maintain structural integrity of the evaporative element when it is subjected to prolonged use in water, heat and air flow circulation. Any material conventionally used as an absorbing material is used in the present invention. Other examples of wicking materials include sponge, mesh or other porous materials. Further, coatings are known that aid water retention and distribution on non-porous surfaces.

Any material conventionally used for wicking is used as a wicking material in the present invention. For example, the wick comprises a web comprising a plurality of fibers and a coating overlaying at least a portion of a plurality of the fibers. In one embodiment, the coating comprises a plurality of organic particles, the fibers define a plurality of pores and the web may define a plurality of macropores. The fibers of the web are woven or non-woven. The web comprises a nonwoven macroporous material.

It is desirable to minimize the countertop space dedicated to the device and unnecessary to include the means to eject the writing instrument or stylus in situations where a writing instrument or stylus is not used. Further, it remains desirable to provide a means to disinfect a portion of one's hand and to not spread contamination in situations where a keypad, touch screen, handle, or other shared surface is used.

Another embodiment of the present invention is directed toward a disinfecting device for a portion of the user's hand. The disinfecting device comprises: (i) a germicide comprising an amount effective to disinfect the finger(s) or hand, (ii) a support base having a cavity within the support base for containing the germicide and an opening in a surface of the base, and (iii) a germicidal carrier for retaining germicide and carrying it to the opening such that at least a portion of the finger(s) or hand will contact the germicide upon touching the germicidal carrier. In this embodiment, the device itself, or when removably seated within a fixed holding fixture, can be mounted to any surface at any angle.

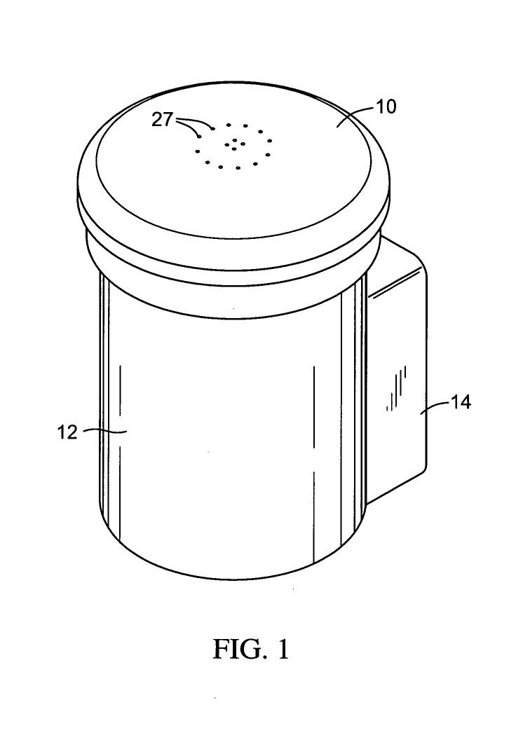

FIG. 1 represents an external view of an embodiment of a device for disinfecting a portion of a user's hand, comprising a base 12, an external membrane 10, perforations 27, and an external flat for surface mounting 14. The rear surface of the external flat 14, while shown essentially vertical in FIG. 1, could be manufactured at any angle for mounting to non-vertical surfaces.

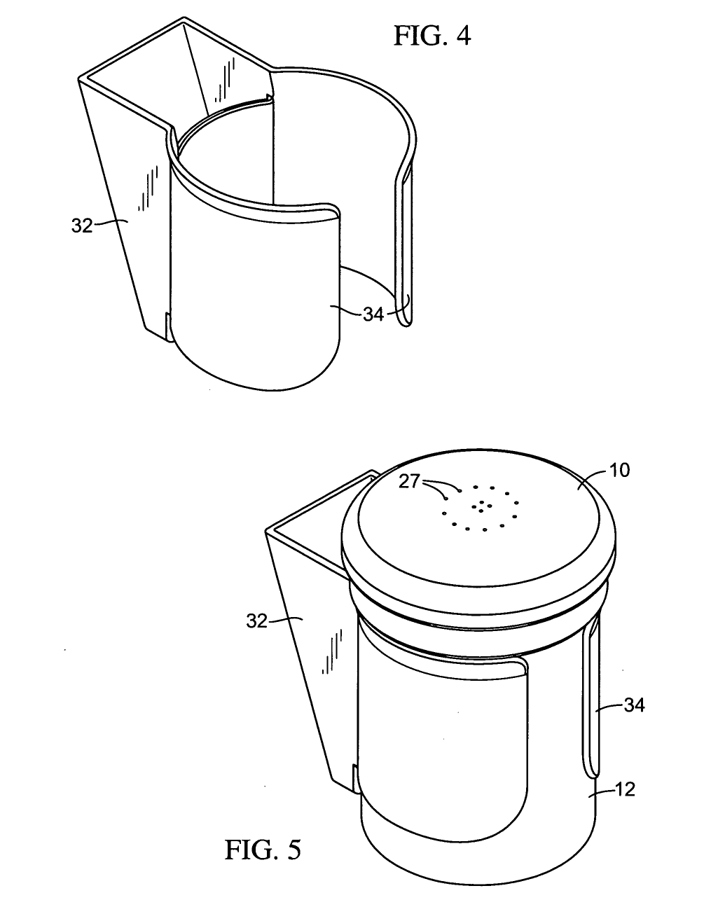

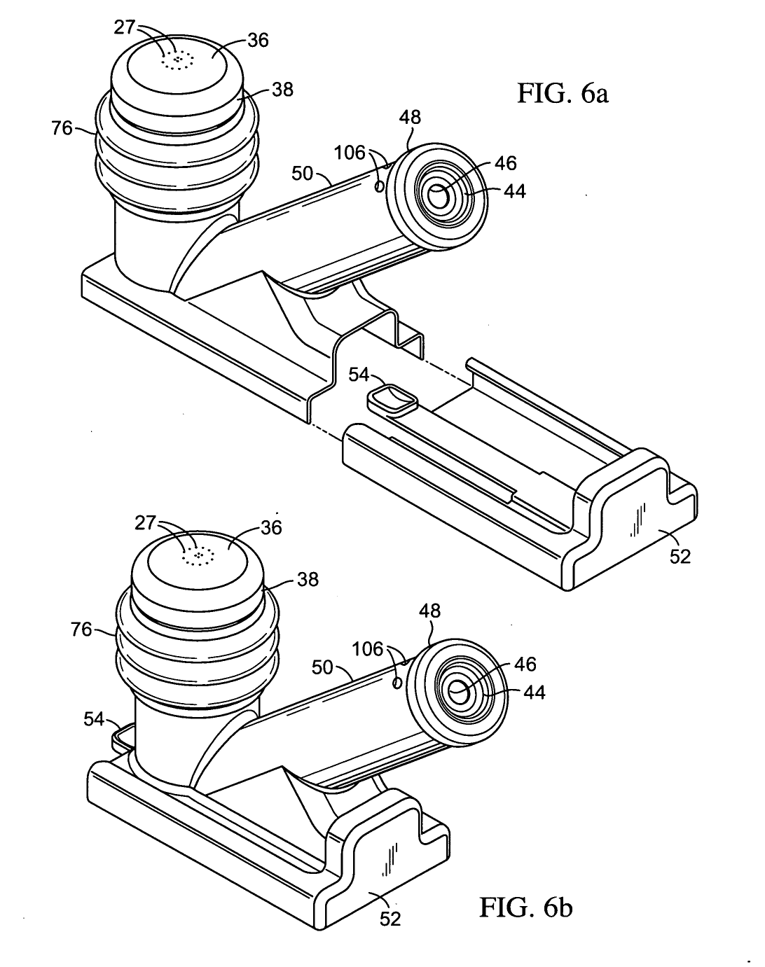

An alternate embodiment of a mounting system of the finger disinfecting device depicted in FIG. 1 is represented in FIG. 4 and FIG. 5. FIG. 4 illustrates an angled support mounting 32. This angled mounting support 32 could be manufactured at any angle for mounting to either vertical or non-vertical surfaces. Retentive support arms 34 engage an undercut in the base 12, and constitute a means to removably retain the device within the support mounting 32. In this embodiment, the support bracket mounting assembly, consisting of the angled support mounting 32 and the retentive support arms 34, substitute for the external flat for surface mounting 14.

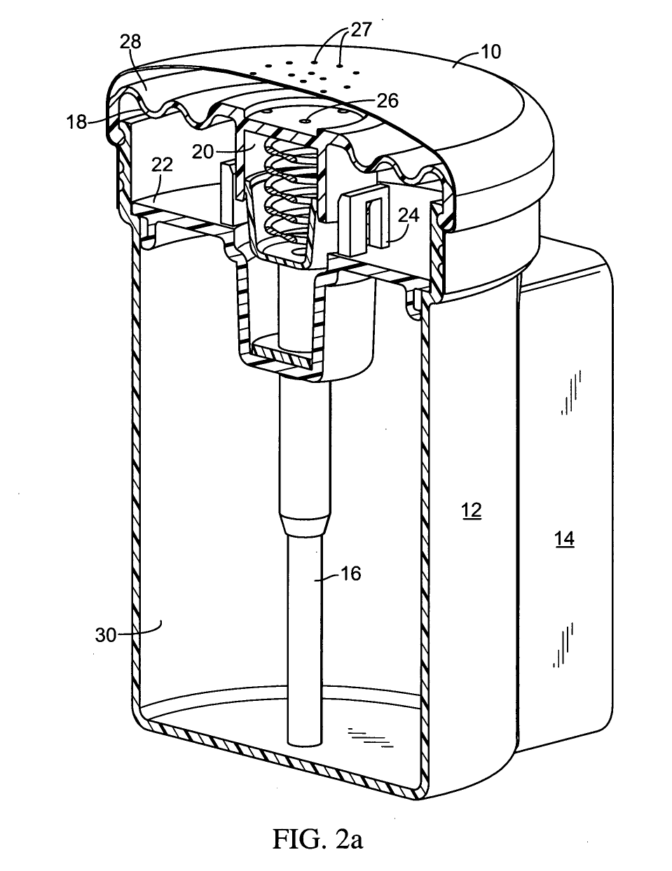

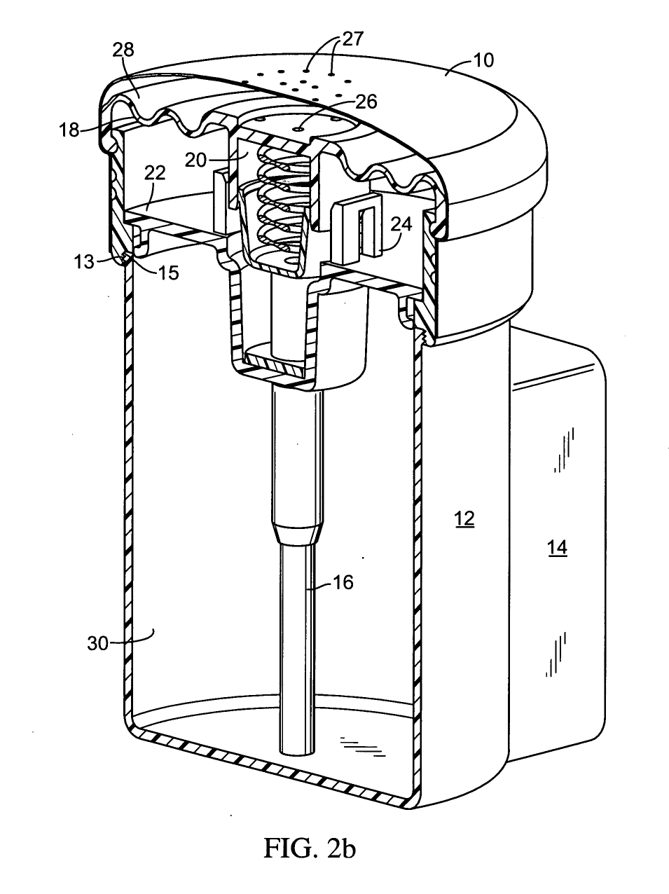

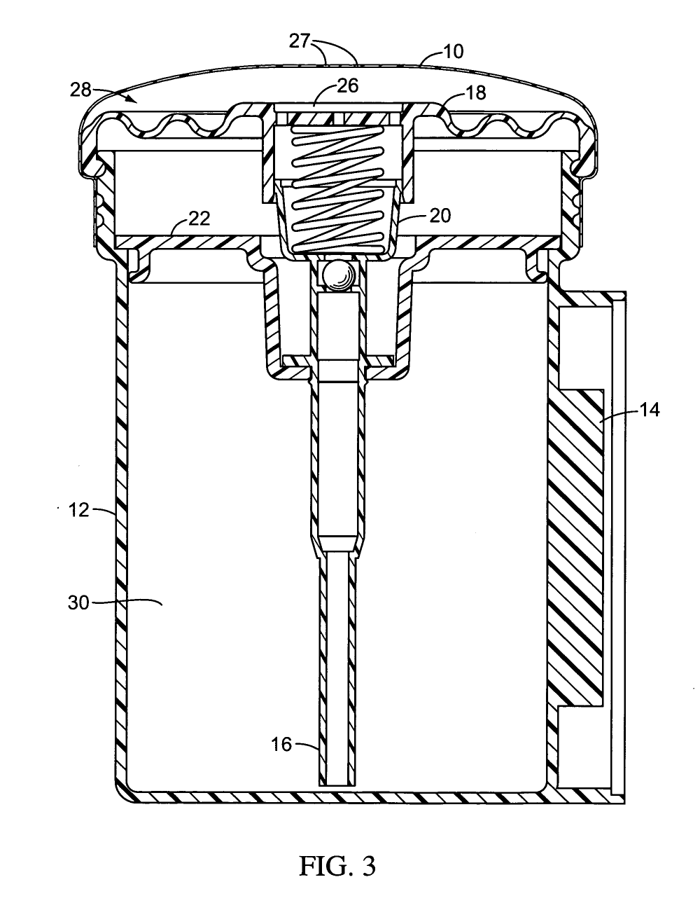

FIG. 2a is a representative cross section (cutaway) view of a disposable embodiment of the hand portion disinfecting device. FIG. 2b is a representative cross section (cutaway) view of a refillable embodiment of the hand portion disinfecting device. FIG. 2b illustrates the mating internal threading 13 of the base 12, and the external threading 15 of the upper assembly (10,18,20,22,24,26,27,28). FIG. 3 is a representative cross-section of an embodiment of the hand portion disinfecting device rotated approximately 45 degrees along the vertical axis from the representation depicted in FIG. 2a. The external membrane 10 has perforations 27 or microperforations in its central portion to permit the flow of germicide 116 through the external membrane 10. In preferred application the external membrane 10 would be a nonabsorbent, nonreactive material and feel slippery or slick to the user. Teflon® based materials e.g. Gore-Tex®. or Teflon®/silicone materials e.g. Silon® are envisioned as membrane materials but are selectively permeable materials and would be additionally perforated to permit adequate flow of germicide 116 past the membrane interface. The base 12 holds a reservoir of germicide 30. An aerosol spray pump head 20 is held in position between the dispenser support 22 and a corrugated cap 18. The central section of the corrugated cap 18 includes a flattened central area with one or more minute perforations constituting a spray orifice 26. The corrugations provide a degree of flexibility of movement, which would be present utilizing either a plastic or elastomeric material. The corrugated cap 18 snaps into place surrounding the spray pump head 20, and a fluid resistant seal may be further developed along this junction, as necessary, with the use of lubricants or sealants. Locking tabs 24 engage mating protrusions and further stabilize and secure the relative positions of 20, 22 and 24 within the body of the base 12. A siphon tube 16 extends from the spray pump head 20 to the bottom of the germicide reservoir 30. The spray head 20 and siphon tube 16 assembly is a germicide delivery device capable of dispensing substantially all of the germicidal product within the base 12. Absorbent material 28 is located between the external membrane 10 and the corrugated cap 18.

The device represented in FIGS. 1-5 is used by applying downward force onto the perforated external membrane 10 which in turn: a) compresses the absorbent material 28, b) exudes any part of the germicide 30 held within the absorbent material 28 through the perforations 27 of the external membrane 10, c) compresses the spring mechanism of the spray pump head 20, which d) draws additional germicide 30 from the reservoir upward and through the spray orifice 26. A percentage of the germicide 30 exiting the spray orifice 26 will immediately pass through the perforations 27 and further wet the applied finger(s) with germicide 30. The wetting of the external membrane 10, could be varied by a) adding additional perforations to the external membrane 10, and/or b) substituting a common mechanical foam dispensing head for the spray pump head 20. The remaining percentage of germicide 30 will be retained between the external membrane 10 and the corrugated cap 18, and become absorbed with the absorbent material 28. Further, the corrugated cap 18 is capable of flexure, and formed of a flexible, resilient material such as silicone rubber, other rubber or plastic, etc. The central portion of the corrugated cap 18, housing the spray head 20, is substantially rigid, and capable of reliably engaging the locking tabs 24. In most embodiments the germicide 30 is volatile, and will evaporate from the absorbent material 28. When the downward force is released, the spray pump head 20 and the corrugated cap 18 will return to their original positions. The user may elect to repeat the above sequence to gain additional protection from contamination prior to or subsequent to using a keypad, touch screen, or other fixture.

The movement of the corrugated cap 18, as described, is determined by the applied force, the flexure of the materials used and the number and thickness of the corrugations. As illustrated in FIG. 2a, the perimeter of the corrugated cap 18 is fixably engaged to the base 12. Vertical movement of the corrugated cap 18 could also be achieved by having the perimeter of the corrugated cap 18 disengaged and moving freely alongside the base 12.

As described above, the present digit device is an aerosol delivery system. Other pressurized, foam, or gravity-fed delivery systems are included within the scope of the present invention and are claimed herein.

The shape of the devices is not a complex geometry, which contributes to the ease with which they may be cleaned. Additionally, the devices may be fabricated from available bacteria resistant materials.

While the principles of the invention have been made clear in illustrative embodiments, there will be immediately obvious to those skilled in the art many modifications of structure, arrangement, proportions, the elements, materials, and components used in the practice of the invention, and otherwise, which are particularly adapted to specific environments and operative requirements without departing from those principles.

Specifically, the figures depict embodiments generally placed adjacent to or approximate to areas of intended usage, e.g. one use of the angled writing instrument device illustrated in FIGS. 6a-13 is envisioned placed beside an existing credit card transaction device. These embodiments are “first generation” embodiments of the claims and invention. “Second generation” embodiments include incorporating and integrating the devices into the actual design and manufacture of the underlying related systems. For example, a “second generation” credit card transaction device includes a germicidal component and integrated mechanics to decontaminate the associated writing instrument and keypad.

The appended claims are intended to cover and embrace any and all such modifications, with the limits only of the true purview, spirit and scope of the invention.

The present invention can be practiced by employing conventional materials, methodology and equipment. Accordingly, the details of such materials, equipment and methodology are not set forth herein in detail. In the previous descriptions, numerous specific details are set forth, such as specific materials, structures, chemicals, processes, etc., in order to provide a thorough understanding of the present invention. However, it should be recognized that the present invention can be practiced without resorting to the details specifically set forth. In other instances, well known processing structures have not been described in detail, in order not to unnecessarily obscure the present invention.

Only exemplary embodiments of the present invention and but a few examples of its versatility are shown and described in the present disclosure. It is to be understood that the present invention is capable of use in various other combinations and environments and is capable of changes or modifications within the scope of the inventive concept as expressed herein.

1-63. (canceled)

64. An apparatus for supporting and disinfecting a handheld instrument comprising:
(a) a receptacle, having a cavity, for supporting a handheld instrument;
(b) a germicide; and
(c) a germicide carrier, for carrying the germicide, disposed within the cavity such that a portion of the handheld instrument contacts the germicide carrier and the germicide when the handheld instrument is supported by the receptacle.

65. The apparatus according to claim 64, wherein a cross-section of an inner portion of the receptacle has a cylindrical or ovoid shape.

66. The apparatus according to claim 64, wherein the receptacle stands substantially upright.

67. The apparatus according to claim 64, wherein the receptacle includes a diaphragm for partially covering an opening to the cavity, the diaphragm including an opening sized such that the handheld instrument may be inserted into said receptacle.

68. The apparatus according to claim 67, comprising an abrasive ring proximal to the diaphragm.

69. The apparatus according to claim 67, comprising a ring having a scent-producing material proximal to the diaphragm.

70. The apparatus according to claim 64, wherein the receptacle includes at least one vent hole for evaporation of liquid.

71. The apparatus according to claim 64, wherein the germicidal carrier has a shape such that the germicide contacts substantially all of an outer peripheral surface of the portion of the handheld instrument.

72. The apparatus according to claim 64, wherein the germicidal carrier comprises an absorbent material for retaining the germicide.

73. The apparatus according to claim 72, wherein said absorbent material comprises a sponge, mesh or porous material for wicking the germicide to the germicide carrier.

74. The apparatus according to claim 64, further comprising a base for supporting said receptacle.

75. The apparatus according to claim 64, comprising a reservoir for containing said germicide, the reservoir communicating with said receptacle to allow for the absorption of germicide into the germicidal carrier.

76. The apparatus according to claim 64, wherein the apparatus further comprises an ejector for expelling the handheld instrument.

77. The apparatus according to claim 76, wherein said ejector is electrically operated.

78. The apparatus according to claim 76, wherein said ejector comprises a lever for expelling the handheld instrument when the lever is operated.

79. The apparatus according to claim 78, wherein said lever comprises a cam shaped protrusion for contacting said receptacle.

80. The apparatus according to claim 64, wherein the germicide comprises an alcohol.

81. The apparatus according to claim 64, wherein the germicide comprises at least one of iodophors, peracids, hypochlorites, chlorine dioxide, quaternary ammonia compounds, u.v. light, and ozone.

82. The apparatus according to claim 64, wherein the germicide comprises at least one of a liquid germicide, a powder germicide, a solid germicide, a gas germicide, a gel germicide or a light source.

83. The apparatus according to claim 64, wherein the handheld instrument comprises a writing device, a signature device or an indicating device.

84. The apparatus according to claim 64, further comprising a second germicide carrier for contacting and carrying the germicide, and for contacting a portion of a hand for disinfecting the hand portion with the germicide.

85. The apparatus according to claim 84, comprising a perforated membrane for contacting the germicide carrier and the hand portion such that the germicide is transferred from the germicide carrier to the hand portion.

86. The apparatus according to claim 84, wherein said second germicide carrier is for wicking germicide from said reservoir.

87. The apparatus according to claim 84, wherein said second germicide carrier comprises an ejector for expelling the handheld instrument.

88. The apparatus according to claim 75, further comprising a status indicator, wherein said status indicator comprises a sensor to detect the amount of said germicide in the reservoir.

89. The apparatus according to claim 86, wherein said status indicator detects the purity of said germicide.

90. The apparatus according to claim 86, wherein said status indicator detects a concentration of said germicide.

91. The apparatus according to claim 86, wherein said status indicator indicates an elapsed time since said germicide was added to said reservoir.

92. The apparatus according to any one of claims 86-91, wherein said status indicator depicts a status of a property of said germicide as a visual or an auditory output.

93. The apparatus according to claim 75, wherein said reservoir is refillable with additional germicide.

94. The apparatus according to claim 75, wherein said reservoir further comprises a removable package containing said germicide.

95. The apparatus according to claim 94, wherein said removable package comprises a sealed flexible container.

96. The apparatus according to claim 74, wherein said base is detachable from said receptacle.

97. The apparatus according to claim 93, wherein said reservoir comprises an opening for refilling said germicide.

98. The apparatus according to claim 64, further comprising a germicide delivery device for contacting and delivering the germicide to a portion of a hand for disinfecting the hand portion with the germicide.

99. The apparatus according to claim 98, comprising a membrane for contacting the germicide delivered by the germicide delivery device and the hand portion such that the germicide is transferred to the hand portion.

100. The apparatus according to claim 99, wherein the germicide delivery device comprises a pump operatively connected to the membrane for transferring the germicide from the reservoir to the membrane.

101. An apparatus comprising:
a base having a cavity and an opening at a surface of the base;
a germicide disposed in the cavity; and
a germicide carrier for carrying the germicide to the opening for contacting a portion of a hand for disinfecting the hand portion with the germicide.

102. The apparatus according to claim 101, comprising a perforated membrane at the opening for contacting the germicide carrier and the hand portion such that the germicide is transferred from the germicide carrier to the hand portion.

103. The apparatus according to claim 102, wherein the base further comprises a reservoir for containing said germicide, the reservoir communicating with said cavity to allow for the absorption of germicide into the germicide carrier.

104. The apparatus according to claim 103, comprising a pump operatively connected to the membrane for transferring the germicide from the reservoir to the germicide carrier.

105. The apparatus according to claim 103, wherein said reservoir is refillable with additional germicide.

106. The apparatus according to claim 103, wherein said reservoir further comprises a removable package containing said germicide.

107. The apparatus according to claim 106, wherein said removable package comprises a sealed flexible container.

108. The apparatus according to claim 64, wherein the receptacle comprises a credit card transaction device.

109. The apparatus according to claim 101, wherein the base comprises a button panel assembly.

110. The apparatus according to claim 64, wherein the receptacle comprises a touchscreen device.

111. The apparatus according to claim 64, wherein the receptacle comprises a keypad device.

112. The apparatus according to claim 64, wherein the receptacle comprises a biometric or personal identification recognition device.

113. The apparatus according to claim 101, wherein the base comprises a credit card transaction device.

114. The apparatus according to claim 101, wherein the base comprises a touchscreen device.

115. The apparatus according to claim 101, wherein the base comprises a keypad device.

116. The apparatus according to claim 101, wherein the base comprises a biometric or personal identification recognition device.

117. An apparatus comprising:
a base having a cavity and an opening at a surface of the base;
a germicide disposed in the cavity; and
a germicide delivery device to deliver the germicide to the opening for contacting a portion of a hand and for disinfecting the hand portion with the germicide.

118. The apparatus according to claim 117, comprising a membrane overlying the opening for contacting the hand portion and disinfecting the hand portion with the germicide.

119. The apparatus according to claim 118, wherein the base further comprises a reservoir for containing said germicide, the reservoir communicating with said cavity to allow for the delivery of germicide to the membrane.

120. The apparatus according to claim 119, comprising a pump operatively connected to the membrane for transferring the germicide from the reservoir to the membrane.

121. The apparatus according to claim 103, wherein said reservoir is refillable with additional germicide.

122. The apparatus according to claim 103, wherein said reservoir further comprises a removable package containing said germicide.

123. The apparatus according to claim 122, wherein said removable package comprises a sealed flexible container.

124. The apparatus according to claim 121, wherein said reservoir comprises an opening for refilling said germicide.

125. The apparatus according to claim 119, wherein said reservoir is refillable with additional germicide.

126. The apparatus according to claim 119, wherein said reservoir further comprises a removable package containing said germicide.

127. The apparatus according to claim 126, wherein said removable package comprises a sealed flexible container.

128. The apparatus according to claim 125, wherein said reservoir comprises an opening for refilling said germicide.
